(19) United States
(12) Patent Application Publication
(10) Pub. No.: US 2005/0266041 A1
(43) Pub. Date: Dec. 1, 2005

(54) IMPLANT FOR VESSEL LIGATURE

(75) Inventors: Bodo Gerold, Himmelstadt (DE); Claus Harder, Uttenreuth (DE); Heinz Mueller, Erlangen (DE); Bernd Heublein, Hannover (DE); Eva Heublein, Hannover (DE); Nora Heublein, Hannover (DE); Christoph Heublein, Hannover (DE)

Correspondence Address: HAHN LOESER & PARKS, LLP, One GOJO Plaza, Suite 300, AKRON, OH, 44311-1076, US

(73) Assignee: RESTATE PATENT AG, Baar/Zug (CH)

(21) Appl. No.: 10/908,729

(22) Filed: May 24, 2005

(30) Foreign Application Priority Data
May 25, 2004 (DE) 102004026104.0

(51) Int. Cl. A61F 2/06; A61F 2/00

(52) U.S. Cl. 424/423; 623/1.12

(57) ABSTRACT

The invention concerns inter alia an implant for vessel ligature, the implant which comprises an alloy, wherein the alloy is at least partially biodegradable, and wherein the alloy comprises: greater than 87% magnesium; from about 3% to about 6% yttrium; from about 1% to about 5% lanthanide; and a balance of about 0.0% to about 2%.

This application claims the benefit of German patent application serial number 10 2004 026 104.0, filed May 25, 2004, which is hereby incorporated by reference.

### TECHNICAL FIELD

The invention concerns an implant for vessel ligature.

### BACKGROUND OF THE INVENTION

The term vessel ligature is used in the medical art to denote tying off or tying up a blood vessel, which is effected at the location of choice or at the location of an also operative wound. Vessel ligature possibly also includes stitching through or around the vessel. To carry out the operation, the operator makes use of various auxiliary means which, inter alia, also include implants which remain in the body of the patient. By way of example of such implants for such vessel ligature, mention may be made of treating aneurysms on the one hand by means of a metal clip or on the other hand by the placement of a coil in the interior of the vessel.

Implants for vessel ligature, which are known from the state of the art, generally comprise biocompatible but not biodegradable materials. In particular, plastic materials and metals together with alloys thereof are suitable as such materials. In spite of all endeavors to improve the compatibility of the implants, the permanent presence of the implants in the human or animal body is often the starting point for complications such as chronic inflammation.

In addition, nuclear magnetic resonance tomography is in the meantime a daily routine in terms of radiological diagnostics. In that case, the patient is exposed both to strong statistical and also variable weaker magnetic fields. In the presence of implants of ferromagnetic materials, they are attracted by the magnetic field, moved and in the worst-case scenario removed from their original position. In addition, serious image defects (artifacts) can occur on the sectional views of an examination due to the metals. Depending on the respective extent of such artifacts, the response to the diagnostic question involved can be made more difficult or in an extreme case can even be made completely impossible. Implants of plastic material can admittedly eliminate those effects, but they generally involve material properties which are unsuitable for such an application.

The presence of the implants is frequently only necessary for a limited period of time from a medical point of view, either due to the vessel growing permanently shut naturally after closure thereof or due to termination of the healing process in the part of the blood vessel which is only closed for a short time. The implant which has become redundant in such a situation can only be removed by a fresh operation.

DE 101 28 100 discloses a medical implant for the human and animal body, which at least partially comprises a biodegradable magnesium alloy which contains proportions of rare earth metals and lithium. Reference is made inter alia to use as a surgical suture material, in particular as wound clips. The magnesium alloy preferably contains proportions of lithium of 0-7%, aluminum of 0-16%, rare earth metals of 0-8% and yttrium of 0-7%. The rare earth metal can be neodymium.

EP 1 270 023 also discloses coils and clips comprising a metallic material which is degradable in vivo. The metallic material is an alloy, the main constituent of which can be an alkaline earth metal, in particular magnesium.

A biodegradable material which is suitable for use in implants for vessel ligature should satisfy a number of requirements:
it should enjoy the mechanical properties necessary for production and use of the implant (workability, breaking strength, etc.),
the material itself and also it degradation products should be physiologically completely harmless, and
in vivo degradation should occur uniformly and controllably and should be adapted to the respective application.

The search for a suitable material is correspondingly complicated and expensive. All previously known solutions have hitherto not led to a satisfactory result.

### SUMMARY OF THE INVENTION

Accordingly the object of the present invention is inter alia to provide a material suitable for implants for vessel ligature.

It has now surprisingly been found that implants for vessel ligature, the implant which comprises an alloy, wherein the alloy is at least partially biodegradable, and wherein the alloy comprises:
greater than 87% magnesium;
from about 3% to about 6% yttrium;
from about 1% to about 5% lanthanide; and
a balance of about 0.0% to about 2%
   having particularly good material properties for the specified purposes of use and the degradation products even have a positive physiological effect on the surrounding tissue. All particulars relating to the alloy are in percent by weight, the individual components of the alloy totaling 100%.

Extremely good properties for the material and highly promising physiological effects in respect of the degradation products can preferably be achieved on the one hand with an alloy with the yttrium present at a concentration of about 3.7% to about 4.3% and a lanthanide present at a concentration of about 2.4% to about 4.4% and an alloy with the yttrium present at a concentration of about of 4.75% to about 5.5% and a lanthanide present at a concentration of about of 1.5% to about 4%.

In another embodiment of the present invention, the lanthanide further comprises neodymium. It has surprisingly been found that neodymium, at a concentration of 2% to about 2.5% in the alloy, improves the physiological compatibility of the alloy and its degradation products. In addition, the alloy exhibits material properties which are particularly suitable for production and use of implants for vessel ligature, in particular clips and coils, such as non-ferromagnetic characteristics, ease of deformability and adequate breaking strength.

In another embodiment of the present invention, the balance further comprises elements selected from the group consisting of lithium, zirconium and zinc, wherein the lithium is at a concentration of about 0.15% to about 2%, the zirconium is at a concentration of about 0.4% to about 1% and the zinc is at a concentration of about 0.004% to about 0.2%. The presence of the specified elements considered in themselves or in any combination evidently has an influence on the mechanical properties of the implant. Thus in particular in vivo degradation of the implant is also dependent on the predetermined proportion of lithium. Furthermore the presence of zirconium leads to a marked reduction in stress crack corrosion.

### DETAILED DESCRIPTION OF THE INVENTION

Degradation performance of the implant can preferably be predetermined in dependence on an alloy morphology, a thickness of material of the implant and the alloy composition. The term “degradation performance” is used to denote the degradation of the alloy according to the invention, in the living organism, which takes place over time due to chemical, thermal, oxidative, mechanical or biological processes. On the one hand the aim is to ensure that, at least in the first weeks after implantation, the desired medical-technical properties of the implant which are generally determined by its mechanical integrity are retained. On the other hand the aim is that the presence of rigid structures which can be the starting point of a whole cascade of rejection reactions is maintained only over a period of time which is absolutely necessary.

Preferably, the degradation performance of the implant is predetermined in such a way that 80% by weight or more of the implant, with respect to the total weight of the alloy present in the implant, is degraded in a period of time of between about 6 months and about 10 years. A further aspect of the invention is that the degradation performance of the implant is to be predetermined in such a way that its mechanical integrity is maintained for at least 4 and in particular 6 months. In that respect the term “mechanical integrity” is used to denote the stability, which is still sufficient in spite of progressive degradation, of the structure elements of the implant, which are necessary to fulfill the medical purpose of the implant, that is to say tying up or tying off a vessel. In other words, degradation can accordingly already have resulted admittedly in degradation of a considerable part of the implant, but precisely not the part thereof which is necessary to safeguard the medical purpose.

Degradation of the implant in the above-indicated sense can be delayed for example by increasing the thickness of material. Equally, method steps governed by the production process have an influence on degradation (thus cast implants generally degrade more quickly than extruded implants). In addition, an increase in the proportion of lithium results in delayed degradation. The complexity of in vivo degradation which depends not just on the configuration of the implant and the morphology and composition of the material used but also the position of the implant in the body means that it is necessary in each case to determine the degradation performance of an implant intended for specific purposes, on a situation-related basis.

It has further surprisingly been found that extruded alloys have improved physiological properties in comparison with cast alloys. The physiological properties are thus at least in part governed by the method of manufacture. Thus conventional cell tests on untreated smooth human muscle cells exhibited pronounced proliferation inhibition in the presence of the alloy according to the invention and its degradation products. The precise physiological active mechanism here is hitherto not been clarified.

Table 1 set out hereinafter shows two examples for alloys suitable for the manufacture of clips or coils:

TABLE 1

| | | | | | | L | | |
|---|---|---|---|---|---|---|---|---|
| No. | Zn | Li | Zr | Y | Nd | with Nd | further | Mg |
| 1 | 0.1 | 0.15 | 0.55 | 4.1 | 2.2 | 3.1 | 0.4 | 91.6 |
| 2 | 0.2 | 0.2 | 0.7 | 5.1 | 2.0 | 2.8 | 0.2 | 90.8 |

The particulars relating to the components of the alloys relate to percentages by weight. ‘L’ stands for lanthanides and ‘further’ stands for other elements which are combined in the alloy component balance, such as silicon, copper, manganese, iron, nickel and silver. The amounts were determined with a degree of accuracy of about +/−0.1%.

The foregoing description of embodiments of the present invention has been provided for the purposes of illustration and description. It is not intended to be exhaustive or to limit the invention to the precise forms disclosed. Obviously, many modification and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

1. An implant for vessel ligature, the implant comprising:
an alloy, wherein the alloy is at least partially biodegradable, and wherein the alloy comprises:
greater than 87% magnesium;
from about 3% to about 6% yttrium;
from about 1% to about 5% lanthanide; and
a balance of about 0.0% to about 2%.

2. The implant of claim 1, wherein the yttrium in the alloy is present at a concentration of about 3.7% to about 4.3% and the lanthanide in the alloy is present at a concentration of about 2.4% to about 4.4%.

3. The implant of claim 1, wherein the yttrium in the alloy is present at a concentration of about 4.75% to about 5.5% and the lanthanide in the alloy is present at a concentration of about 1.5% to about 4.0%.

4. The implant of claim 1, wherein the lanthanide further comprises neodymium.

5. The implant of claim 2, wherein the lanthanide further comprises neodymium.

6. The implant of claim 4, wherein the neodymium is present at a concentration of about 2% to about 2.5%.

7. The implant of claim 1, wherein the balance further comprises elements selected from the group consisting of lithium, zirconium and zinc.

8. The implant of claim 3, wherein the balance further comprises elements selected from the group consisting of lithium, zirconium and zinc.

9. The implant of claim 5, wherein the balance further comprises elements selected from the group consisting of lithium, zirconium and zinc.

10. The implant of claim 7, wherein the lithium is present at a concentration of about 0.15% to about 0.2%.

11. The implant of claim 7, wherein the zirconium is present at a concentration of about 0.4% to about 1.0%.

12. The implant of claim 7, wherein the zinc is present at a concentration of about 0.004% to about 0.2%.

13. The implant of claim 1, wherein the implant is a clip or a coil.

14. The implant of claim 6, wherein the implant is a clip or a coil.

15. The implant of claim 1, wherein a degradation performance of the implant is at least 80% by weight of the implant, with respect to the total weight of the alloy present in the implant, is degraded in a period of time of between at least 6 months to about 10 years.

16. The implant of claim 7, wherein a degradation performance of the implant is at least 80% by weight of the implant, with respect to the total weight of the alloy present in the implant, is degraded in a period of time of between at least 6 months to about 10 years.

17. The implant of claim 12, wherein a degradation performance of the implant is at least 80% by weight of the implant, with respect to the total weight of the alloy present in the implant, is degraded in a period of time of between about 6 months to about 10 years.

18. The implant of claim 1, wherein a degradation performance of the implant provides evidence that a mechanical integrity is maintained for at least about 4 months.

19. The implant of claim 11, wherein a degradation performance of the implant provides evidence that a mechanical integrity is maintained for at least about 4 months.

20. The implant of claim 13, wherein a degradation performance of the implant provides evidence that a mechanical integrity is maintained for at least about 4 months.
